(19) United States
(12) Patent Application Publication
(10) Pub. No.: US 2013/0084545 A1
(43) Pub. Date: Apr. 4, 2013

(54) Pressure Wave Root Canal Cleaning System

(71) Applicant: BIOLASE, INC., Irvine, CA (US)

(72) Inventors: Vladimir Netchitailo, Livermore, CA (US); Dmitri Boutoussov, Dana Point, CA (US); Vladimir Lemberg, Santa Clara, CA (US); Rudolf Marius Verdaasdonk, Houten (NL)

(73) Assignee: BIOLASE, INC., Irvine, CA (US)

(21) Appl. No.: 13/632,628

(22) Filed: Oct. 1, 2012

Related U.S. Application Data
(60) Provisional application No. 61/541,743, filed on Sep. 30, 2011.

(51) Int. Cl. A61C 5/02

(52) U.S. Cl. 433/224

(57) ABSTRACT

Systems and methods are provided for cleaning or disinfecting a target region. A fluid including a plurality of gas bubbles is placed into an interaction zone. The interaction zone is a volume that extends into the target region or that is adjacent to the target region. The fluid in the interaction zone is exposed to electromagnetic radiation, where the electromagnetic radiation has a wavelength that is substantially absorbed by the fluid. The fluid in the interaction zone substantially absorbs the electromagnetic radiation to create an acoustic shock wave and a pressure wave. The acoustic shock wave and the pressure wave cause a movement of the fluid and cavitation effects that are configured to clean or disinfect the target region.

### CROSS-REFERENCE TO RELATED APPLICATIONS

This application claims priority to U.S. Provisional Patent Application No. 61/541,743, filed Sep. 30, 2011, entitled “Carbonation-Stimulated Liquid Cleaning System,” which is herein incorporated by reference in its entirety.

### TECHNICAL FIELD

The technology described herein relates generally to electromagnetic radiation emitting devices and more particularly to the use of electromagnetic radiation emitting devices for cleaning or disinfecting a cavity, canal, or surface.

### BACKGROUND

A primary cause of infection, disease, and death in humans is inadequate bacteria control. Thus, killing or removing bacteria from various systems of the human body is an important part of many medical and dental procedures. For example, during a root canal procedure, the root canal is cleaned by mechanical debridement of the canal wall and an application of an antiseptic substance within the canal to kill some of the remaining bacteria. However, dental technology has found it difficult to completely eradicate all bacteria during a root canal procedure. In particular, the structural anatomy of the tooth makes it difficult to eliminate all bacteria because the root canal includes irregular lateral canals and microscopic tubules where bacteria can lodge and fester. Bacteria control in other medical and dental procedures has proven equally difficult, and the failure to control bacteria during these procedures can lead to a variety of health and medical problems (e.g., presence of bacteria in the bloodstream, infection of organs including the heart, lung, kidneys, and spleen).

Outside of the medical and dental fields, control of bacteria or other foreign matter (e.g., dirt, particulate matter, adhesives, biological matter, residues, dust, stains) in various systems is also important. For example, cleaning and disinfection of toys, eating utensils, and other objects with which humans come in contact may be an important way of reducing the spread of illness. Further, cleaning and removal of various substances from surfaces and openings may also be pursued for aesthetic reasons (e.g., restoration of artwork).

### SUMMARY

Systems and methods are provided for cleaning or disinfecting a target region. In a method for cleaning or disinfecting a target region, a fluid including a plurality of gas bubbles is placed into an interaction zone. The interaction zone is a volume that extends into the target region or that is adjacent to the target region. The fluid in the interaction zone is exposed to electromagnetic radiation, where the electromagnetic radiation has a wavelength that is substantially absorbed by the fluid. The fluid in the interaction zone substantially absorbs the electromagnetic radiation to create an acoustic shock wave and a pressure wave. The acoustic shock wave and the pressure wave are configured to cause a movement of the fluid and cavitation effects that are configured to clean or disinfect the target region.

A system for cleaning or disinfecting a target region includes a fluid having a plurality of gas bubbles. The fluid is placed into an interaction zone, where the interaction zone is a volume that extends into the target region or that is adjacent to the target region. The system also includes an electromagnetic energy source configured to produce electromagnetic radiation having a wavelength that is substantially absorbed by the fluid. The electromagnetic radiation is used to expose the fluid in the interaction zone, and the fluid substantially absorbs the electromagnetic radiation to create an acoustic shock wave and a pressure wave, and the acoustic shock wave and the pressure wave cause a movement of the fluid and cavitation effects configured to clean or disinfect the target region.

### BRIEF DESCRIPTION OF THE FIGURES

FIGS. 1A, 1B, 1C, and 1D depict an example system for cleaning or disinfecting a target region.

FIG. 2 depicts a block diagram of an example system utilizing an electromagnetic energy source to clean or disinfect a target region.

FIG. 3 depicts example liquid cleaning systems with a fiber optic tip being placed in different locations relative to a canal.

FIG. 4 depicts self-centering fiber optic tip systems used to center a fiber optic tip within a canal or near an entrance to the canal.

FIG. 5 depicts example timing diagrams illustrating aspects of a method for cleaning or disinfecting a target region with a fluid including a plurality of gas bubbles.

FIG. 6 depicts fiber optic cables inserted into root canals of a tooth for intra-canal disinfection or cleaning

FIG. 7 depicts an example method for cleaning a target region that utilizes abrasive materials to aid in the cleaning

FIG. 8 is a flowchart illustrating an example method for cleaning or disinfecting a target region.

### DETAILED DESCRIPTION

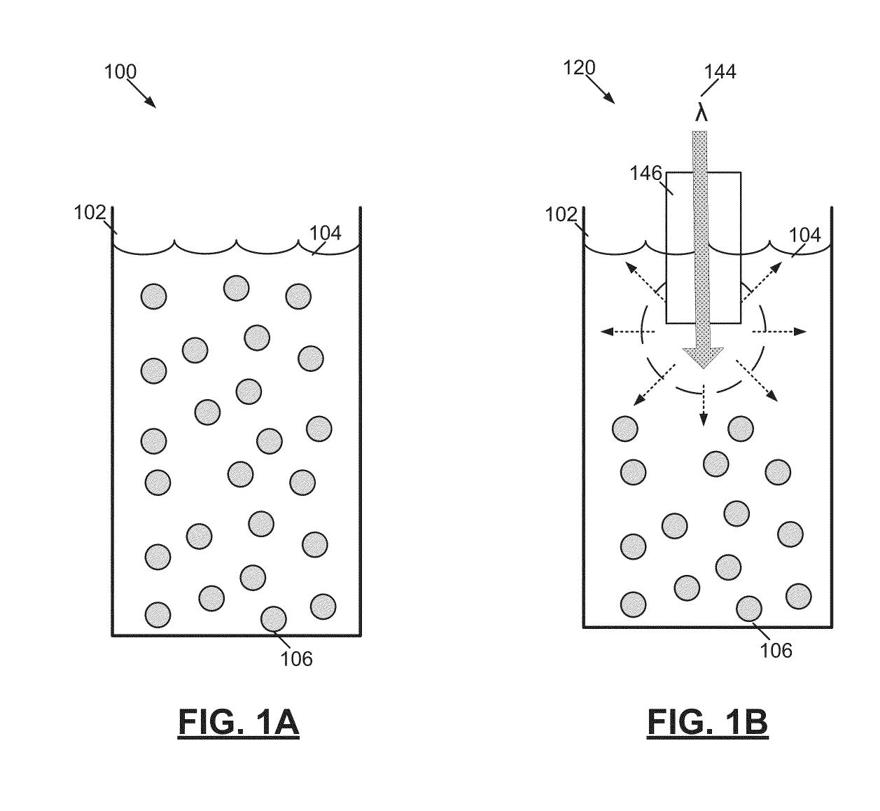

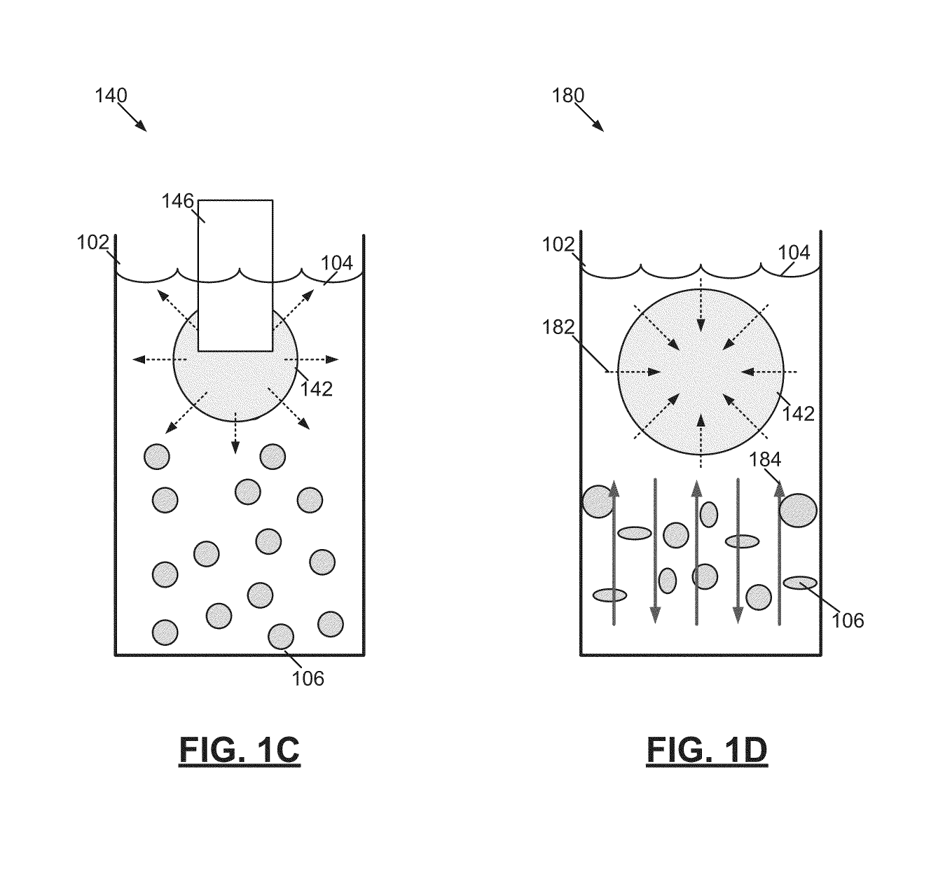

FIGS. 1A, 1B, 1C, and 1D depict an example system for cleaning or disinfecting a target region 102. FIG. 1A depicts the example system during a first period of time 100. In FIG. 1A, a fluid 104 including a plurality of gas bubbles 106 is placed within the target region 102. The fluid 104 may be a carbonated fluid (e.g., sparkling water, carbonated soft drink, beer, champagne, or another fluid containing a similar concentration of gas bubbles), a nitrogenated fluid, a hydrogenated fluid, or a fluid containing bubbles of another composition (e.g., gas bubbles containing a medication or a bacteria-killing substance such as iodine). Such fluid has a bubble concentration of approximately 1,000 to 100,000,000 bubbles per liter. The gas bubbles 106 have diameters ranging from approximately 0.1 μm to 500 μm and can be identified as micro-bubbles (diameters 0.1 μm to 5 μm) and macro-bubbles (diameters 5 μm to 500 μm). The fluid 104 may be, for example, a water-based solution or a saline solution. A difference between a regular fluid and the fluid 104 as used in the example of FIGS. 1A, 1B, 1C, and 1D is the concentration of the micro-bubbles, which is higher in the fluid 104 versus the regular fluid by several orders of magnitude. In the fluid 104, micro-bubbles may be combined with other micro-bubbles to form the macro-bubbles. The target region 102 is a cavity, canal, passage, opening, or surface which is to be cleaned or disinfected (e.g., a root canal including bacteria or other matter to be removed from the root canal).

FIG. 1B depicts the example system during a second period of time 120. During the second period of time 140, a fiber optic tip 146 is positioned within the target region 102. The fiber optic tip is an electromagnetic radiation emitting fiber optic tip and is connected via a fiber optic cable to an electromagnetic energy source. The electromagnetic energy source generates electromagnetic radiation 144 that is routed along the fiber optic cable and emitted by the fiber optic tip 146. The electromagnetic radiation 144 emitted by the fiber optic tip 146 has a wavelength that is substantially absorbed by the fluid 104. The fiber optic tip 146 may be of a variety of different shapes (e.g., conical, angled, beveled, double-beveled), sizes, designs (e.g., side-firing, forward-firing), and materials (e.g., glass, sapphire, quartz, hollow waveguide, liquid core, quartz silica, germanium oxide). In one example, the fiber optic tip 146 is made of water-free silica glass material with a diameter of 400 μm. Further, although the system of FIGS. 1A, 1B, 1C, and 1D illustrates the use of the fiber optic tip 146 as the light emitting element of the system, in other examples, various waveguides, light emitting elements (e.g., light emitting nanoparticles and nanostructures), or devices including mirrors, lenses, and other optical components may be used in place of the fiber optic tip 146 for light emission.

As explained in further detail below, the electromagnetic radiation 144 emitted by the fiber optic tip 146 is absorbed by the fluid 104, which causes an acoustic shock wave and pressure waves to be created in the fluid 104. These waves generate a movement of the fluid 104 (i.e., a high-speed fluid motion) that is used to clean or disinfect the target region 102. The acoustic shock waves can cause effective disruptive and cleaning actions due to non-linear mechanical effects (e.g., cavitations, turbulence, and microjets). Micro-bubbles of the fluid 104 amplify an efficiency of the process. During the second period of time 120, the acoustic shock waves are generated because of a rapid energy absorption in a small volume of liquid. The rapid energy absorption in the small volume of liquid creates huge thermo-elastic stresses and leads to generation of the acoustic shock waves that spread through the volume of the fluid 104 and interact disruptively with the target region 102. These waves are capable of killing bacteria and removing any contaminations from the surfaces of the target region 102. The acoustic shock waves may have characteristic times of a few microseconds.

During the third period of time 140, a vapor bubble 142 is created within the fluid 104. The vapor bubble 142 is created by the exposure of the fluid 104 to the electromagnetic radiation 144 at the wavelength that is substantially absorbed by the fluid 104. Due to the high absorption of the electromagnetic radiation 144 in the fluid 104, the vapor bubble 142 forms near the end of the fiber optic tip 146. Pressure waves generated by an expansion and collapse of the vapor bubble 142 cause compression and deformation of the bubbles 106 and additional movement of the fluid 104 that contributes to further cleaning or disinfecting of the target region 102. The pressure waves are related to liquid displacement stimulated by expansion and collapse of the vapor bubble 142 and have characteristic times of approximately 100 microseconds.

As noted above, the fluid 104 is configured to substantially absorb the electromagnetic radiation 144. In FIG. 1B and 1C, the fluid 104 is water-based, and the electromagnetic radiation has a wavelength in the range of approximately 1.8 μm-10.6 μm or 0.157 μm-300 μm, which is substantially absorbed in the water-based fluid 104. In one example, the electromagnetic radiation 144 is delivered to the fluid 104 as a pulse of light, with a wavelength in a range of approximately 1.8 μm-10.6 μm or 0.157 μm-300 μm, a pulse duration in a range of 0.5 μs-10 ms, a pulse energy of 20 mJ, and an average pulse power of 200 W.

During a fourth period of time 180, after reaching its maximum diameter, the vapor bubble 142 collapses, as indicated by the inward-pointing arrows 182. The collapsing of the vapor bubble 142 includes a rapid implosion, with the implosion creating pressure waves in the fluid 104. The pressure waves create high-speed fluid motion 184 in the fluid 104. The pressure waves incident on the gas macro-bubbles 106 of the fluid 104 compress at least some of the gas macro-bubbles 106, and following the compression, the gas macro-bubbles 106 expand, as illustrated in FIG. 1C. In particular, the gas bubbles 106 increase a compressibility of the fluid 104, such that the pressure waves created by the explosive vapor bubble 142 can have a greater effect throughout the entirety of the target region 102 and not only within the vicinity of the vapor bubble 142 itself. In systems lacking the gas macro-bubbles 106, the fluid 104 is a uniform and less compressible fluid, and pressure waves created by an explosive vapor bubble 142 have less of an effect in the target region 102 as the distance from the vapor bubble 142 increases. Thus, use of the fluid 104 with the gas macro-bubbles 106 may allow achievement of the fluid motion effect while the fiber optic tip 146 is placed near a top of the target region 102, rather than at a greater depth in the target region 102, which may help to prevent the fiber optic tip 146 from breaking in the target region 102.

The use of the gas bubbles 106 in the fluid 104 within the target region 102 decreases a threshold amount of energy needed for generation of the acoustic shock waves and increases an efficiency of disruptive interaction with the target region 102. Deformation of the gas bubbles 106 during action of the pressure waves also improves a capability of fluid 104 to flow and remove contaminations out of the target region 102.

The high-speed fluid motion 184 in the fluid 104 generated by the acoustic shock waves and the explosive vapor bubble create cavitations, turbulences, microjets, and implosions, which are responsible for cleaning or disinfecting the target region 102. In an example system, the high-speed fluid motion 184 created by the pressure waves and the compression and expansion of the gas bubbles 106 is used to remove or kill bacteria from within the target region 102. In another example, the cavitations and implosions created by the high-speed fluid motion 184 may rupture the membranes of cells and pull cells from a dentine matrix of a tooth. Such cells and bacteria may react to the acoustic shock wave and pressure waves in a manner similar to that of the gas bubbles 106 and may undergo compression and expansion. In some examples, the compression and expansion or the impact from the forces of the pressure waves may be enough to kill the cells and bacteria. Thus, the acoustic shock waves, pressure waves, and the high-speed fluid motion 184 may be used as part of an endodontic procedure to disrupt or kill intratubular bacteria or bacteria residing on surfaces of the target region 102.

The target region 102 may be of a small size (e.g., on the order of the size of the fiber optic tip 146) and may be a cavity, canal, passage, opening, or surface of the human body (e.g., a root canal passage, tubule of a tooth, tooth cavity, blood vessel). Thus, the system of FIGS. 1A, 1B, 1C, and 1D for cleaning or disinfecting a target region 102 using an electromagnetic energy source may be employed in the context of a variety of medical or dental procedures (e.g., treating tissue, removing deposits and stains from surfaces, removing or killing bacteria). For example, the system of FIGS. 1A, 1B, 1C, and 1D may be used as part of a root canal treatment procedure, where the high-speed fluid motion 184 is used to clean the root canal, remove or kill bacteria in the root canal, or apply a medical treatment to the root canal. Non-dental applications of the system of FIGS. 1A, 1B, 1C, and 1D include procedures within a human body passage, such as a vessel (e.g., a blood vessel) or an opening, cavity, or lumen within hard or soft tissue (e.g., treatment of occluded arteries or necrotic bone). Another use of the system of FIGS. 1A, 1B, 1C, and 1D is in the treatment of a surface condition of the skin (e.g., skin having an acne condition).

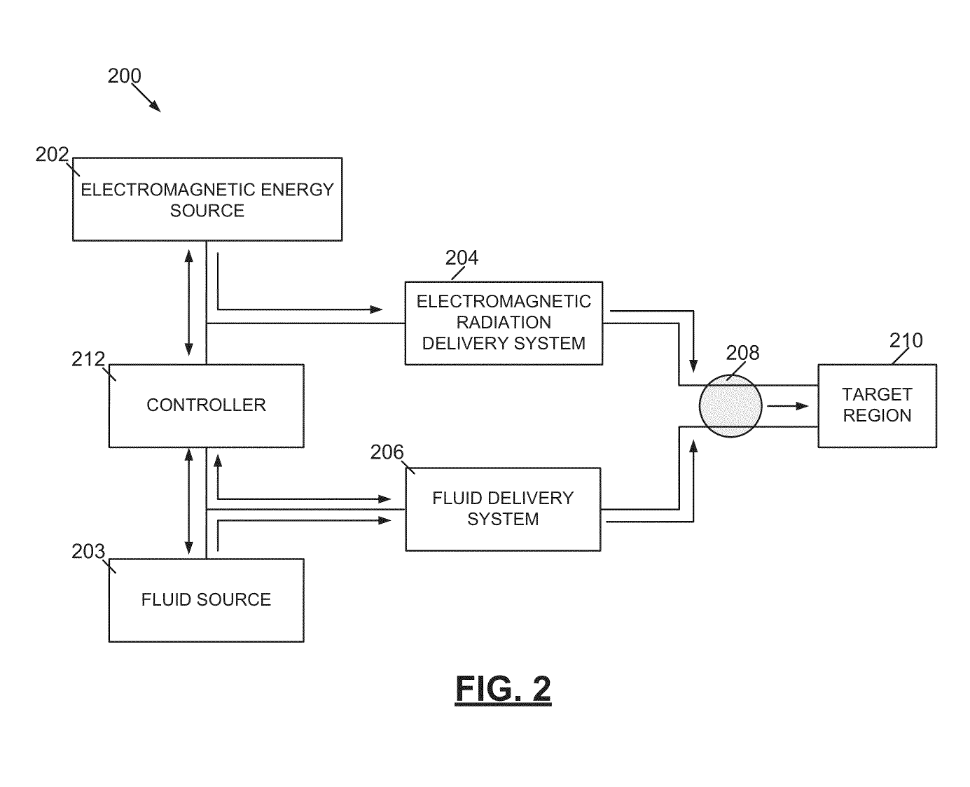

FIG. 2 depicts a block diagram of an example system 200 utilizing an electromagnetic energy source 202 to clean or disinfect a target region 210. In the system 200 of FIG. 2, the electromagnetic energy source 202 is configured to generate electromagnetic radiation at a particular wavelength that is highly absorbed by a fluid used in the system 200. With reference to FIGS. 1B, 1C, and 1D the electromagnetic radiation at the particular wavelength is used to expose the fluid 104 to create the acoustic shock and pressure waves for cleaning or disinfecting the target region 102. The electromagnetic energy source 202 is connected to both an electromagnetic radiation delivery system 204 and a controller 212. The electromagnetic radiation delivery system 204 is configured to emit the electromagnetic energy at the particular wavelength. The electromagnetic radiation delivery system 204 is connected to an interaction zone 208 (e.g., positioned within the interaction zone 208) and focuses or places a peak concentration of the electromagnetic radiation at the particular wavelength onto the fluid within the interaction zone 208. In an example system, the electromagnetic radiation delivery system 204 includes a fiber optic cable and a fiber optic tip. In this system, the fiber optic cable routes the electromagnetic radiation generated by the electromagnetic energy source 202 to the fiber optic tip for emission into the interaction zone 208. In another example system, the fiber optic tip and the fiber optic cable are not used, and the fluid in the interaction zone 208 is exposed to the electromagnetic radiation via another means (e.g., a waveguide, light emitting nanoparticle or nanostructure, quantum dot, or devices including mirrors, lenses, and other optical components). The interaction zone 208 is a volume of space that extends into the target region 210 or that is adjacent to the target region 210. Further, with reference to FIGS. 1B, 1C, and 1D the interaction zone 208 includes an area in which the electromagnetic radiation emitted from the electromagnetic radiation delivery system 204 and the fluid interact to form the acoustic shock and pressure waves in the fluid.

The interaction zone 208 is also connected to a fluid delivery system 206, which is configured to supply the fluid to the interaction zone 208. The fluid delivery system 206 receives the fluid from a fluid source 203. In one example, the fluid delivery system 206 is configured to fill the volume comprising the interaction zone 208 with the fluid. The interaction zone 208 may be a portion of a cavity, opening, canal, or passage, and the fluid delivery system 206 may be configured to fill the portion of the cavity, opening, canal, or passage with the fluid. The fluid may be a carbonated fluid containing carbon dioxide bubbles (e.g., sparkling water, carbonated soft drink, beer, champagne, or another fluid containing a similar concentration of gas bubbles) or may be a non-carbonated fluid containing a plurality of nitrogen bubbles or bubbles of another composition (e.g., gas bubbles containing a medication or a bacteria-killing substance such as iodine).

The controller 212 is connected to the electromagnetic energy source 202, the fluid source 203, and the fluid delivery system 206, and is used to synchronize the delivery of the electromagnetic radiation and the fluid to the interaction zone 208. The fluid may be delivered to the interaction zone 208 prior to the delivery of the electromagnetic radiation or may be delivered simultaneously with the radiation. In addition to synchronizing the delivery of the electromagnetic radiation and the fluid to the interaction zone 208, the controller 212 also controls various operating parameters of the electromagnetic energy source 202, the fluid source 203, and the fluid delivery system 206. In an example system, the electromagnetic energy source 202 includes one or more variable wavelength light sources, and the controller 212 allows a user to control the one or more variable wavelength light sources to change the particular wavelength of light emitted by the electromagnetic radiation delivery system 204. The user may change the particular wavelength emitted by the electromagnetic radiation delivery system 204 in order to tailor the emitted wavelength to the absorption properties of the particular fluid used. In another example system, the electromagnetic energy source 202 includes a plurality of light sources. In this example, the system 200 is equipped to work with a larger variety of fluids, and a user selects which of the multiple light sources are to be used via the controller 212.

The electromagnetic energy source 202 may include a variety of different lasers, laser diodes, or other sources of light. The electromagnetic energy source 202 may use an erbium, chromium, yttrium, scandium, gallium garnet (Er, Cr:YSGG) solid state laser, which generates light having a wavelength in a range of approximately 2.70 to 2.80 μm. Laser systems used in other examples include an erbium, yttrium, aluminum garnet (Er:YAG) solid state laser, which generates light having a wavelength of 2.94 μm; a chromium, thulium, erbium, yttrium, aluminum garnet (CTE:YAG) solid state laser, which generates light having a wavelength of 2.69 μm; an erbium, yttrium orthoaluminate (Er:YAL03) solid state laser, which generates light having a wavelength in a range of approximately 2.71 to 2.86 μm; a holmium, yttrium, aluminum garnet (Ho:YAG) solid state laser, which generates light having a wavelength of 2.10 μm; a quadrupled neodymium, yttrium, aluminum garnet (quadrupled Nd:YAG) solid state laser, which generates light having a wavelength of 266 nm; an argon fluoride (ArF) excimer laser, which generates light having a wavelength of 193 nm; an xenon chloride (XeCl) excimer laser, which generates light having a wavelength of 308 nm; a krypton fluoride (KrF) excimer laser, which generates light having a wavelength of 248 nm; and a carbon dioxide (CO2) laser, which generates light having a wavelength in a range of approximately 9.0 to 10.6 μm.

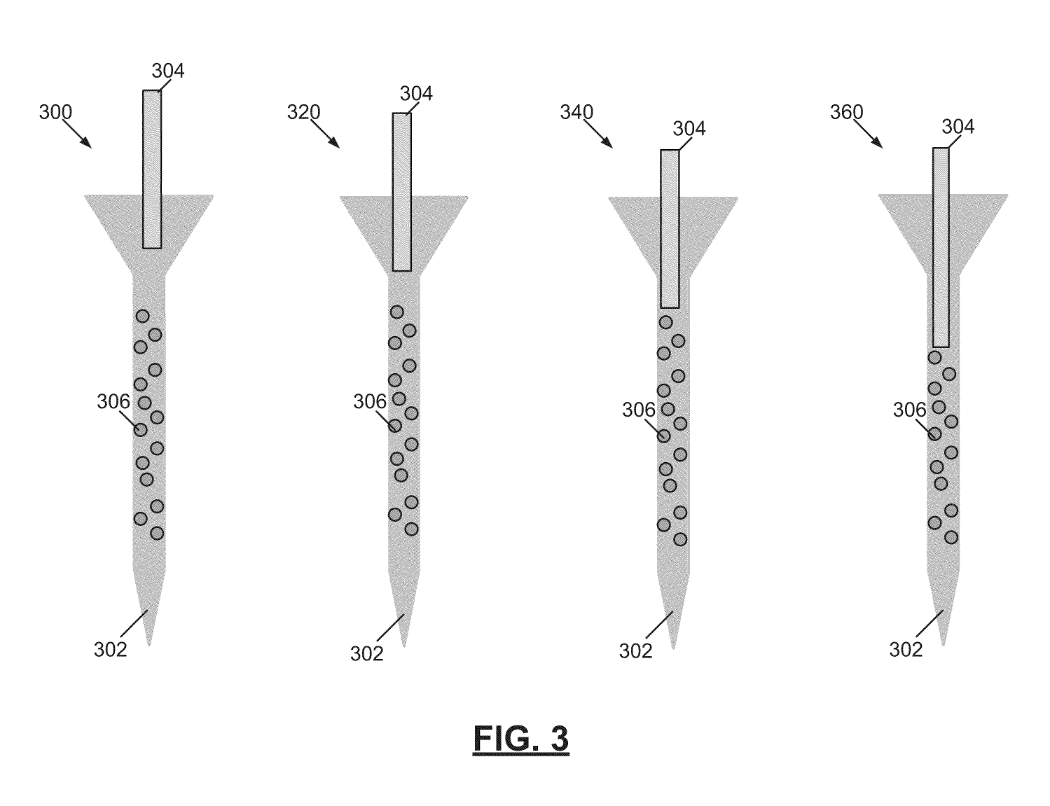

FIG. 3 depicts example liquid cleaning systems 300, 320, 340, 360 with a fiber optic tip 304 being placed in different locations relative to a canal 302. The canal 302 includes a wider opening at the top (e.g., a pulp chamber of a canal in a tooth) and tapers to a thinner diameter near the bottom. In each of the example systems 300, 320, 340, 360, a fluid including gas bubbles 306 is placed into the canal 302. The fiber optic tip 304 is configured to focus or place a peak concentration of electromagnetic radiation onto the fluid 306, where the electromagnetic radiation has a wavelength that is substantially absorbed by the fluid 306. The fluid 306 absorbs the electromagnetic radiation to create a pressure wave that causes high-speed motion of the fluid 306 that is configured to clean the canal 302 or kill bacteria within the canal 302.

The placement of the fiber optic tip 304 in the different locations relative to the canal 302 may affect properties of the high-speed motion of the fluid 306 and properties of the cleaning of the canal 302. In the system 300, the fiber optic tip 304 is placed near the wider opening at the top of the canal 302 and is centered within the wider opening. In the system 320, the fiber optic tip 304 is similarly centered within the canal 302 but is positioned at a deeper position within the wider opening of the canal 302. In the system 340, the fiber optic tip 304 is positioned inside of the main body of the canal 302 at a certain distance (e.g., 2 millimeters), and in the system 360, the fiber optic tip 304 is positioned inside of the main body of the canal 304 at a deeper distance (e.g., 3 millimeters). In each of the example systems 300, 320, 340, 360, the fiber optic tip 304 is not inserted the entire depth of the canal 304, which may help to prevent the fiber optic tip 304 or a fiber optic cable connected to the fiber optic tip 304 from breaking within the canal 302. In each of the systems 300, 320, 340, 360, the canal 302 has dimensions on the order of the size of the fiber optic tip 304.

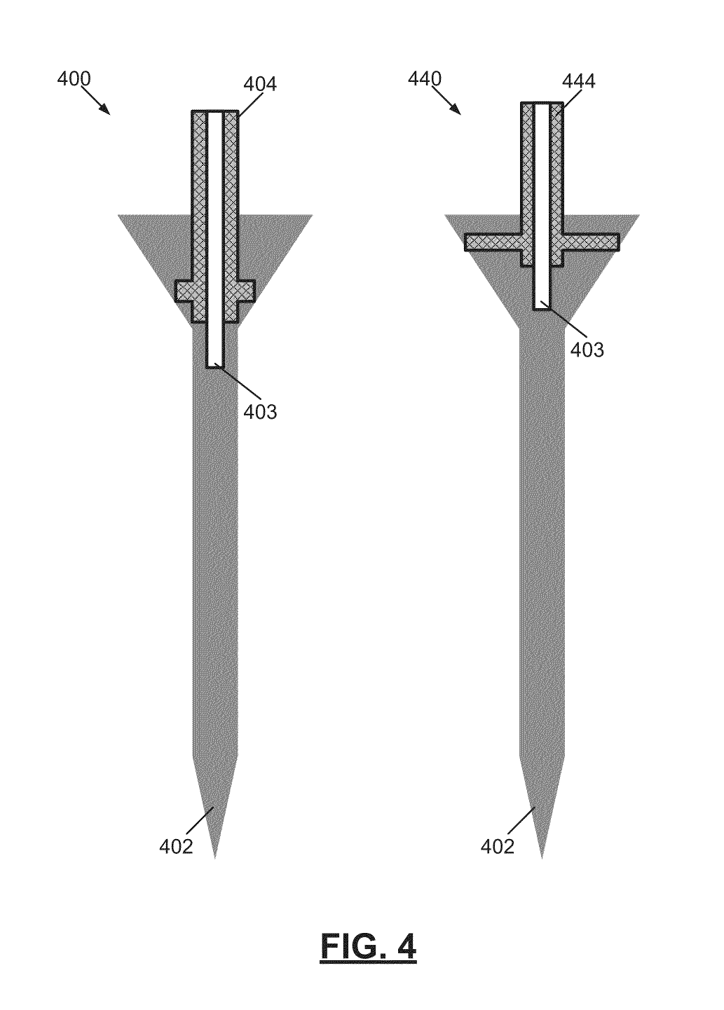

FIG. 4 depicts self-centering fiber optic tip systems 400, 440 used to center a fiber optic tip 403 within a canal 402 or near an entrance to the canal 402. In the system for cleaning or disinfecting a target region described in the preceding figures, it may be desirable to center the fiber optic tip within the cavity, opening, passage, or canal, or to center the fiber optic tip near the entrance to the cavity, opening, passage, or canal. Centering the fiber optic tip in these systems may create an optimal fluid motion for cleaning or disinfecting the cavity, opening, passage, or canal. The self-centering fiber optic tip systems 400, 440 of FIG. 4 are used to achieve this centering of the fiber optic tip 403.

The self-centering fiber optic tip systems 400, 440 utilize cladding layers 404, 444 that fit over a portion of the fiber optic tip 403 and allow the tip 403 to be centered within the canal 402 or near the entrance to the canal 402. The varying thicknesses of the cladding layers 404, 444 between the systems 400, 440 cause the fiber optic tip 403 to be centered at different locations relative to the canal 402. Specifically, the cladding layer 404 of the system 400 allows the tip 403 to be centered within the canal 402, and the cladding layer 444 of the system 440 allows the tip 403 to be centered near the entrance to the canal 402. Other designs may be utilized to create similar self-centering fiber optic tip systems. In one example, the self-centering fiber optic tip system includes a removable band that fits around the fiber optic tip 403 and that serves a similar purpose to the cladding layers 404, 444 of FIG. 4.

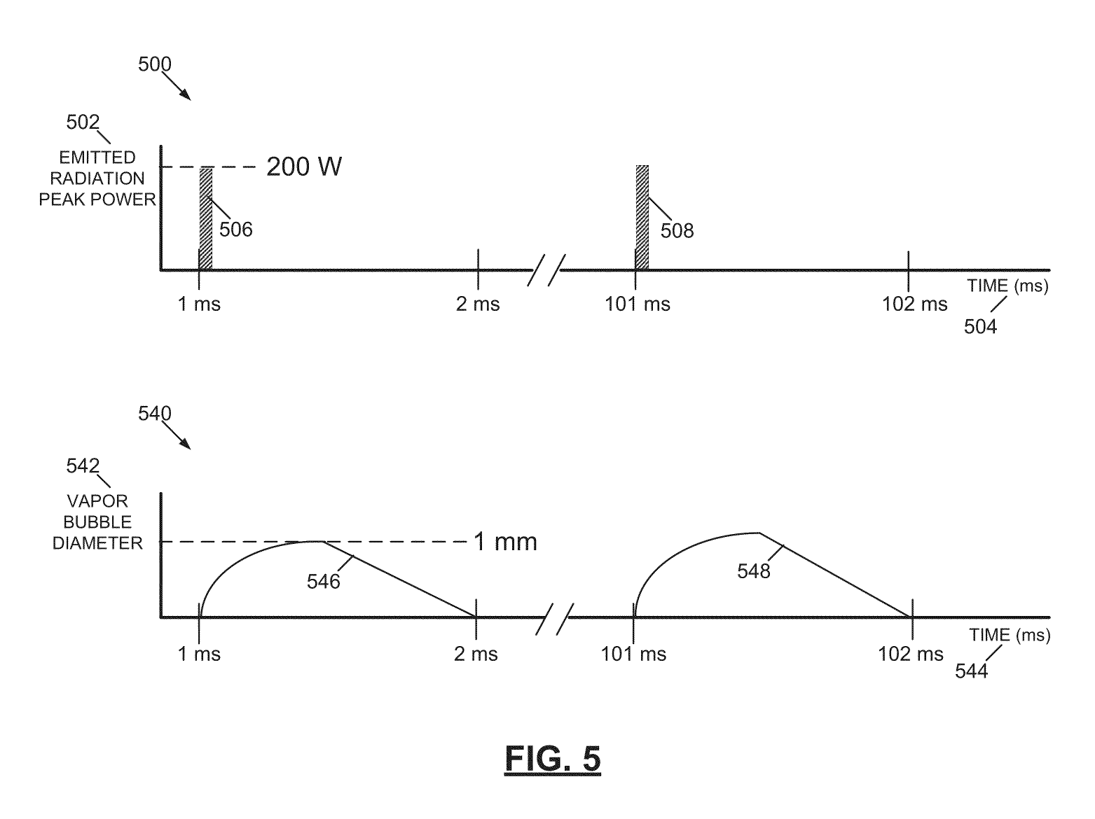

FIG. 5 depicts example timing diagrams 500, 540 illustrating aspects of a method for cleaning or disinfecting a target region with a fluid including a plurality of gas bubbles. Timing diagram 500 is a graph with the X axis representing units of time 504 and the Y axis representing peak power of emitted radiation 502 in watts. With reference to FIG. 1C, the timing diagram 500 illustrates aspects relating to the delivery of the electromagnetic radiation 144, which is used to create the vapor bubble 142 in the fluid 104. At a time of 1 ms, a pulse 506 of the electromagnetic radiation is emitted by the fiber optic tip. The pulse 506 is highly absorbed by a fluid (e.g., the fluid 104 in FIG. 1B) and enables a vapor bubble to form in the fluid. In the timing diagram 500 of FIG. 5, the pulse 506 has a width of 100 μs, a pulse energy of 20 mJ, and a peak power of 200 W. FIG. 5 also depicts a second pulse 508 of the electromagnetic radiation at a time of 101 ms, indicating that pulses of the electromagnetic radiation at the first wavelength are configured to be output at a frequency of 10 Hz (i.e., causing a period of 100 ms between pulses). In an example system, the pulses 506, 508 are delivered at a frequency that matches a resonant frequency of the liquid cleaning system, such that a maximum amount of high-speed fluid motion is created in the fluid for cleaning or disinfecting the target region. In this example, the resonant frequency may be a function of the fluid used in the system, characteristics of the electromagnetic energy source, and of the dimensions of the target region, among other variables.

Timing diagram 540 is a graph with the X axis representing units of time 544 and the Y axis representing a diameter of a vapor bubble 542 in millimeters. With reference to FIG. 1B, the timing diagram 540 illustrates aspects of a bubble cycle of the vapor bubble 142 formed after the fluid 104 is excited by the electromagnetic radiation 144. At a time of 1 ms, in response to the pulse 506 used to excite the fluid, a vapor bubble 546 is created in the fluid. In the timing diagram 540 of FIG. 5, the vapor bubble 546 has a peak maximum diameter of 1 mm and a bubble cycle of approximately 1 ms. As illustrated in the graph 540, upon being exposed to the electromagnetic radiation by the pulse 506, the fluid begins to form the vapor bubble 546. The vapor bubble 546 increases in diameter until it reaches a maximum diameter and then collapses (i.e., rapidly explodes) over the course of the nearly 1 ms bubble cycle. A second bubble 548 is formed in the fluid as a result of the second pulse 508 and has similar characteristics of the first bubble 546. The expansion and collapsing of the vapor bubbles 546, 548 generates a pressure wave in the fluid, and the pressure wave causes a movement (i.e., high-speed motion) of the fluid that is used to clean or disinfect areas of a target region.

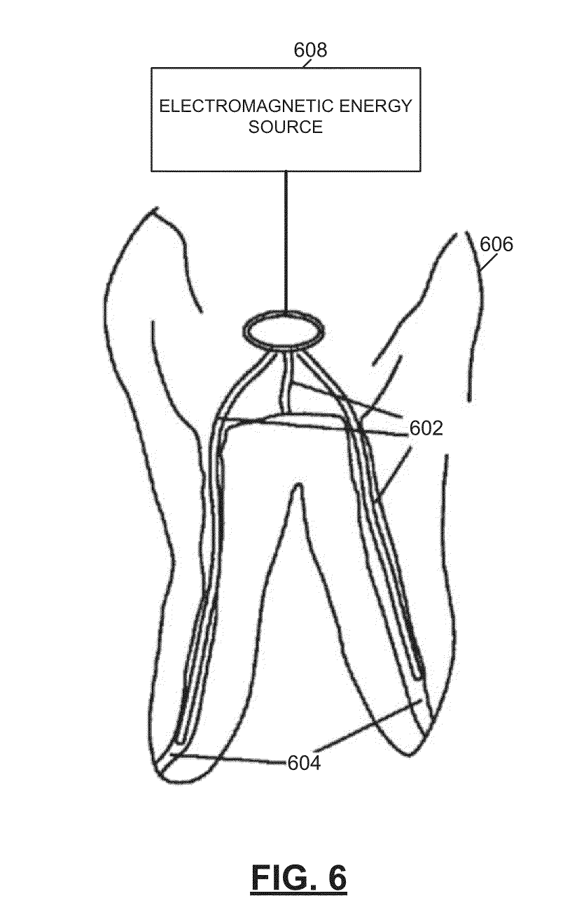

FIG. 6 depicts fiber optic cables 602 inserted into root canals 604 of a tooth 606 for intra-canal disinfection or cleaning The fiber optic cables 602 route electromagnetic radiation from an electromagnetic energy source 608 to fiber optic tips of the cables 602, which extend a substantial distance into the canals 604 in the example of FIG. 6. In other examples, the fiber optic cables 602 are not inserted the substantial distance into the canals 604, and the fiber optic tips are instead positioned near entrances to the canals 604 or inserted a shorter distance into the canals 604 (e.g., as illustrated in FIGS. 3 and 4).

The fiber optic cables 602 may be used with the systems and methods described in the preceding figures to clean or disinfect portions of the tooth 606 or to remove bacteria from the tooth 606. To implement the systems and methods previously described, the canals 604 are filled with a fluid including a plurality of gas bubbles (e.g., a carbonated fluid, fluid containing nitrogen bubbles, or fluid containing bubbles of another composition), and the fiber optic tips of the cables 602 are used to expose the fluid to electromagnetic radiation to create the pressure wave and its associated high-speed fluid motion. In FIG. 6, the target regions to be cleaned via the high-speed fluid motion include various regions within the length of the canals 604. In other examples, the fiber optic cables 602 may be inserted into a tooth cavity or other cavity, opening, or passage of a human body. Such cavities, openings, and passages may have dimensions on the order of the size of the fiber optic cables 602.

Properties of the fiber optic cables 602 and their associated fiber optic tips may be varied to accomplish the cleaning or disinfecting of the target regions. For example, the fibers 602 may include single fibers or multi-fiber bundles of various designs (e.g., radially-emitting tips, side-firing tips, forward-firing tips, beveled tips, conical tips, angled tips). Further, the diameter of the fiber optic cables 602 may be varied, and the cables may have a tapered design with the fiber diameter increasing or decreasing over the length of the cable. The fiber optic tips of the fiber optic cables 602 may be positioned at various distances from the target regions to be cleaned. In certain examples, the fiber optic tips of the fiber optic cables 602 are positioned a number of millimeters from the target region (e.g., positioned a number of millimeters away from the bottom of a canal, where the bottom of the canal is the target region), and in other examples, the fiber optic tips may be positioned directly in contact with the target region (i.e., adjacent to the target region).

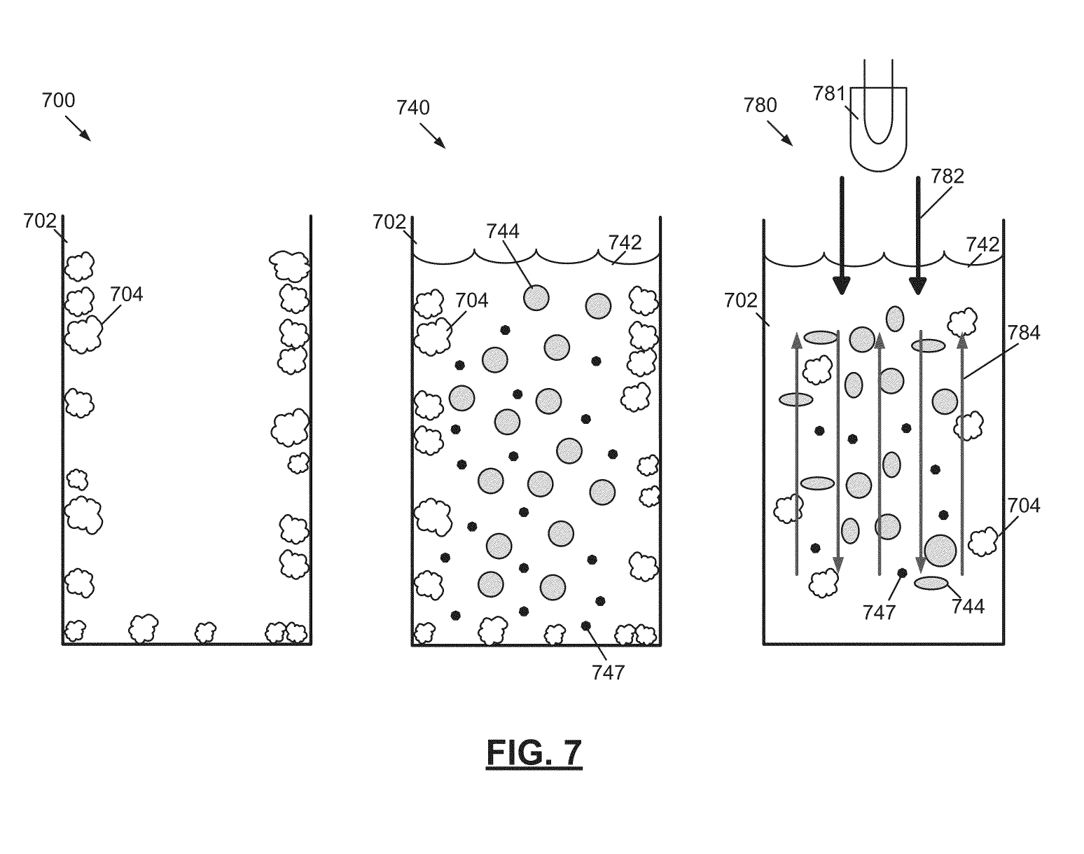

FIG. 7 depicts an example method for cleaning a target region 702 that utilizes abrasive materials 747 to aid in the cleaning The target region 702 of FIG. 7 is a volume, where the volume includes bacteria to be killed or removed or other debris 704 to be removed. Although the target region 702 may be a cavity, opening, passage, canal, or surface of a human or animal body (e.g., a root canal or blood vessel of a human or animal), the target region 702 may also be any type of cavity, opening, passage, canal, or surface that requires disinfection or cleaning In one example, the target region 702 is a portion of a medical or dental device that requires disinfection or cleaning following a use of the device. In another example, the target region 702 is a portion of a microelectronics device or a mechanical device that requires cleaning during construction of the device.

During a first period of time 700, the target region 702 includes the bacteria or debris 704. The debris may include various deposits (e.g. plaque, calculus, dirt, particulate matter, adhesives, biological matter, residue from a cleaning process, dust, stains). Although the bacteria or debris 704 is depicted as being located only on surfaces of the target region 702, in other examples, the bacteria or debris may be located within the inner volume of the target region itself (e.g., suspended within a gas or liquid filling the target region 702).

To remove the bacteria or debris 704 from the target region 702, during a second period of time 740, a liquid 742 including a plurality of gas bubbles 744 is placed into the target region 702. The gas bubbles 744 may be carbon dioxide bubbles, nitrogen bubbles, or gas bubbles of another composition. The gas bubbles of another composition may include gas bubbles of compositions specifically designed for removing the bacteria or debris 704 from the target region 702. For example, iodine gas bubbles may be placed in the target region 702 in order to kill bacteria. Gas bubbles 744 of other compositions may include gas bubbles including medication, such as antibiotics, steroids, anesthetics, anti-inflammatory treatments, antiseptics, disinfectants, adrenaline, epinephrine, astringents, vitamins, herbs, and minerals. The gas bubbles 744 may, for example, have diameters ranging from approximately 0.1 μm to 500 μm.

In addition to the gas bubbles 744, the fluid 742 also includes abrasive materials 747. The abrasive materials 747 are combined with the fluid 742 prior to or after placing the fluid 742 into the target region 702. In other example systems, instead of using the abrasive materials 747, other additional materials combined with the fluid 742 include medications, biologically-active particles, nanoparticles, antiseptics, or antibiotics. The abrasive materials 747 are configured to work with the gas bubbles 744 in removing the bacteria or debris 704 from the target region 702. In one example, the abrasive materials 747 include an aluminum oxide powder having aluminum oxide particles with diameters in a range of approximately 1 μm to 50 μm.

During a third period of time 780, the fluid 742 is exposed to electromagnetic radiation 782, where the electromagnetic radiation 782 has a wavelength that is substantially absorbed by the fluid 742. The electromagnetic radiation 782 is generated by an electromagnetic energy source 781. As illustrated in FIG. 1C, the electromagnetic energy source 781 may be coupled to a fiber optic cable and a fiber optic tip to achieve the exposure of the fluid 742 in the target region 702. Alternatively, the electromagnetic radiation 782 can be delivered to the fluid 742 in a different manner that does not involve fiber optic cables and fiber optic tips. Such alternative methods of delivering the electromagnetic radiation 782 to the fluid 742 can include any system that focuses or places a peak concentration of the electromagnetic radiation 782 onto the fluid 742 in the target region 702. In one example, standard optical lenses configured to focus light are used to expose the fluid 742 to the peak concentration of the electromagnetic radiation 782. The source 781 of the electromagnetic radiation 782 may be a laser, laser diode, lamp, or any other light source configured to produce the electromagnetic radiation 782 having the wavelength that is substantially absorbed in the fluid 742.

The absorption of the electromagnetic radiation 782 by the fluid 742 creates a pressure wave within the fluid 742. The pressure wave causes a high speed motion of the fluid and the gas bubbles 784 that is configured to remove the debris 704 and kill or remove the bacteria 704 from the target region 702. The high speed motion of the fluid and the gas bubbles 784 dissolves the debris 704 and kills or removes the bacteria 704 by imparting strong, concentrated forces onto the debris and bacteria 704. In one example (e.g., as illustrated in FIGS. 1C and 1D), the absorption of the electromagnetic radiation 782 by the fluid 742 creates an explosive vapor bubble that subsequently generates the pressure wave in the fluid 742. The pressure wave causes compression and expansion in at least some of the gas bubbles 744 of the fluid 742. The compression and expansion of the gas bubbles 744 from the pressure waves create turbulence and microjets in the fluid 742, which also contribute to the high-speed fluid motion 784 throughout the target region 702. The abrasive materials 747 are affected by the pressure wave and the high-speed fluid motion 784 and are used to impart forces on the bacteria and debris 704 in order to remove them from the target region 702.

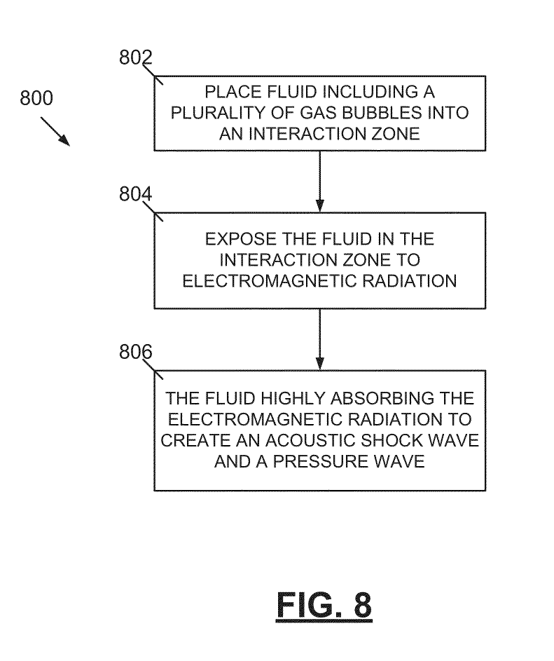

FIG. 8 is a flowchart 800 illustrating an example method for cleaning or disinfecting a target region. At 802, a fluid including a plurality of gas bubbles is placed into an interaction zone. The interaction zone is a volume that extends into the target region or that is adjacent to the target region. At 804, the fluid in the interaction zone is exposed to electromagnetic radiation, where the electromagnetic radiation has a wavelength that is substantially absorbed by the fluid. At 806, the fluid in the interaction zone substantially absorbs the electromagnetic radiation to create an acoustic shock wave and a pressure wave. The acoustic shock wave and the pressure wave cause a movement of the fluid that is configured to clean or disinfect the target region.

While the disclosure has been described in detail and with reference to specific embodiments thereof, it will be apparent to one skilled in the art that various changes and modifications can be made therein without departing from the spirit and scope of the embodiments. Thus, it is intended that the present disclosure cover the modifications and variations of this disclosure provided they come within the scope of the appended claims and their equivalents.

It should be understood that as used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. Further, as used in the description herein and throughout the claims that follow, the meaning of “each” does not require “each and every” unless the context clearly dictates otherwise. Finally, as used in the description herein and throughout the claims that follow, the meanings of “and” and “or” include both the conjunctive and disjunctive and may be used interchangeably unless the context expressly dictates otherwise; the phrase “exclusive of may be used to indicate situations where only the disjunctive meaning may apply.

It is claimed:

1. A method for cleaning or disinfecting a target region, the method comprising:
placing a fluid including a plurality of gas bubbles into an interaction zone, the interaction zone being a volume that extends into the target region or that is adjacent to the target region;
exposing the fluid in the interaction zone to electromagnetic radiation, the electromagnetic radiation having a wavelength that is substantially absorbed by the fluid; and
the fluid in the interaction zone substantially absorbing the electromagnetic radiation to create an acoustic shock wave and a pressure wave, the acoustic shock wave and the pressure wave causing a movement of the fluid and cavitation effects configured to clean or disinfect the target region.

2. The method of claim 1, wherein the absorbing of the electromagnetic radiation creates an explosive vapor bubble in the fluid that expands and collapses, and wherein the expansion and the collapsing of the explosive vapor bubble generates the pressure wave.

3. The method of claim 1, wherein the pressure wave causes a compression and an expansion of macro-bubbles of the gas bubbles, the compression and the expansion of the macro-bubbles and the pressure wave causing the movement of the fluid and the cavitation effects configured to clean or disinfect the target region, and wherein the macro-bubbles have diameters in a range of approximately 5 μm to 500 μm.

4. The method of claim 1, wherein the acoustic shock wave is amplified by micro-bubbles of the plurality of the gas bubbles, and wherein the micro-bubbles have diameters in a range of approximately 0.1 μm to 5 μm.

5. The method of claim 1, further comprising:
positioning an electromagnetic radiation emitting fiber optic tip within the interaction zone, the fiber optic tip being configured to emit the electromagnetic radiation and to execute the exposing of the fluid in the interaction zone to the electromagnetic radiation.

6. The method of claim 5, wherein the target region is a cavity, opening, passage, or canal having dimensions similar in size to dimensions of the fiber optic tip.

7. The method of claim 6, wherein the fiber optic tip is positioned within the interaction zone by placing the fiber optic tip into the cavity, opening, passage, or canal, or by positioning the fiber optic tip near an entrance to the cavity, opening, passage, or canal.

8. The method of claim 7, wherein the fiber optic tip is centered within the cavity, opening, passage, or canal, or is positioned near a center area of the entrance to the cavity, opening, passage, or canal.

9. The method of claim 7, wherein in the placing of the fiber optic tip into the cavity, opening, passage, or canal, the fiber optic tip is not inserted an entire depth of the cavity, opening, passage, or canal.

10. The method of claim 1, wherein the method for cleaning or disinfecting the target region is applied in a medical or dental procedure.

11. The method of claim 1, wherein the cleaning or the disinfecting of the target region includes killing or removing bacteria within the target region or on surfaces of the target region.

12. The method of claim 1, wherein the target region is a root canal passage, tubule of a tooth, tooth cavity, tooth surface, or blood vessel.

13. The method of claim 1, wherein the fluid is water-based, and wherein the wavelength is within a range of approximately 1.8 μm-10.6 μm or 0.157 μm-300 μm.

14. The method of claim 1, wherein the exposing of the fluid in the interaction zone includes exposing the fluid to a pulse of the electromagnetic radiation.

15. The method of claim 14, wherein the exposing of the fluid to the pulse of the electromagnetic radiation includes exposing the fluid to a plurality of the pulses of the electromagnetic radiation, and wherein the plurality of the pulses are delivered at a frequency that matches a resonant frequency of a system including the fluid and the target region.

16. The method of claim 14, wherein the pulse of the electromagnetic radiation has a pulse width within a range of approximately 0.5 μs to 10 ms.

17. The method of claim 14, wherein the pulse of the electromagnetic radiation has a pulse energy within a range of approximately 1 mJ to 250 mJ.

18. The method of claim 1, wherein the gas bubbles of the fluid are carbon dioxide bubbles, nitrogen bubbles, or bubbles of another composition.

19. The method of claim 18, wherein the bubbles of another composition are gas bubbles containing a medication or iodine.

20. The method of claim 1, wherein the gas bubbles of the fluid have diameters in a range of approximately 0.1 μm to 2000 μm.

21. The method of claim 1, wherein the gas bubbles of the fluid have diameters in a range of approximately 0.1 μm to 300 μm.

22. The method of claim 1, further comprising:
combining an abrasive material, nanoparticle, medication, biologically-active particle, antiseptic, or antibiotic with the fluid, the abrasive material, nanoparticle, medication, biologically-active particle, antiseptic, or antibiotic being configured to clean the target region, remove or kill bacteria in the target region, disinfect the target region, or apply a medical treatment to the target region.

23. The method of claim 22, wherein the abrasive material is an aluminum oxide powder having aluminum oxide particles with diameters in a range of approximately 1 μm to 50 μm.

24. The method of claim 1, wherein the exposing of the fluid in the interaction zone includes exposing the fluid to output from an erbium, chromium, yttrium scandium gallium garnet (Er, Cr:YSSG) laser having a wavelength of approximately 2.79 μm.

25. A system for cleaning or disinfecting a target region, the system comprising:
a fluid including a plurality of gas bubbles, the fluid being placed into an interaction zone that is a volume that extends into the target region or that is adjacent to the target region; and
an electromagnetic energy source configured to produce electromagnetic radiation having a wavelength that is substantially absorbed by the fluid, the electromagnetic radiation exposing the fluid in the interaction zone, wherein the fluid in the interaction zone substantially absorbs the electromagnetic radiation to create an acoustic shock wave and a pressure wave, the acoustic shock wave and the pressure wave causing a movement of the fluid and cavitation effects configured to clean or disinfect the target region.
